(19) United States
(12) Patent Application Publication
(10) Pub. No.: US 2009/0069682 A1
(43) Pub. Date: Mar. 12, 2009

(54) SIMPLIFIED CONTROLS FOR IMPLEMENTING DEPTH-BASED GAIN CONTROL IN ULTRASOUND SYSTEMS

(76) Inventors: Harold M. Hastings, Garden City, NY (US); Scott L. Roth, East Hills, NY (US)

Correspondence Address: PROSKAUER ROSE LLP;PATENT DEPARTMENT, 1585 BROADWAY, NEW YORK, NY, 10036-8299, US

(21) Appl. No.: 12/018,621

(22) Filed: Jan. 23, 2008

Related U.S. Application Data
(60) Provisional application No. 60/886,481, filed on Jan. 24, 2007.

(51) Int. Cl. A61B 8/00

(52) U.S. Cl. 600/443

(57) ABSTRACT

An improved approach for implementing depth-based gain control is implemented by adjusting the gain at each depth in the image based on a family of stored curves, with each curve in the family specifying the gain adjustment for all depths as a function of depth. A user interface enables the user to select an entire curve at once (as opposed to the prior art approach of using a set of individually and independently adjustable gain controls for each depth range). The selected curve is then used to modify the gain adjustment provided by the default time gain compensation (TGC) curve.

### CROSS REFERENCE TO RELATED APPLICATIONS

This application claims the benefit of U.S. provisional application No. 60/886,481, filed Jan. 24, 2007, which is incorporated herein by reference.

### BACKGROUND

Conventional ultrasound machines operate by transmitting pulses of ultrasound energy, subsequently receiving ultrasound return signals that have been reflected from relevant anatomical structures, and processing the return signals into images. Due to the finite speed of sound, the return signals that arrive first correspond to shallower depths, and the return signals that arrive later in time correspond to deeper depths. Because ultrasound energy is attenuated both on its way from the transducer to the target region and on its way from the target region back to the transducer, many prior art systems compensate for this attenuation using time gain compensation (TGC) to provide additional amplification for the later-arriving signals, which correspond to deeper depths. Because the roundtrip attenuation of ultrasound energies through many anatomical structures is typically on the order of three decibels per centimeter, a linear gain curve 20 like the one shown in FIG. 1 is often used as the default TGC curve.

However, since the materials through which the ultrasound energy travels are usually not homogenous in real-world applications, the actual attenuation experienced by the ultrasound energy will usually not be a linear function of distance. One known approach for dealing with this nonlinear attenuation is to divide the region being imaged into a number of regions based on depth, and to provide an individual gain adjustment for each region. FIG. 2A is an example of this type of control, in which the region being imaged is divided into eight depths regions A-H, and a slider control 22 is provided to vary the gain independently (above and beyond the gain that is provided by the default TGC curve) in each region. In the illustrated example, the gain in any given region is increased by moving the slider 22 to the right, or decreased by moving the slider 22 to the left.

FIG. 2B is graph of a gain adjustment curve 24 that shows the deviations from the default gain curve. Those deviations are controlled by the positions of the sliders 22 shown in FIG. 2A, which determine how much the gain should be increased or decreased with respect to the default TGC curve. The increase or decrease of gain in each of the depths A-H corresponds to the amount that the slider 22 (shown in FIG. 2A) was moved to the right or left, respectively. To illustrate the mapping between the position of the sliders 22 in FIG. 2A and the gain curve 24 in FIG. 2B, the slider positions 22′ are depicted in dashed lines in FIG. 2B. (Note that the positions of the sliders 22′ in FIG. 2B corresponds to the position of the sliders 22 in FIG. 2A, but rotated 90°.)

After the sliders have been moved to their user-selected positions, conventional ultrasounds systems will vary the gain as a function of depth using an adjusted TGC curve 28, shown in FIG. 2C. The shape of the adjusted TGC curve 28 is based on three components: the default TGC gain 20 (from FIG. 1); the adjustment to that default (based on curve 24, shown in FIG. 2B, which depends on the positions of the sliders); and the contribution of an overall gain control, which boosts the gain by a constant amount over all depths. This overall gain is represented by the offset 26, the magnitude of which is adjustable using any suitable user interface (e.g., a knob or slider control, not shown).

### SUMMARY

The gain in an ultrasound system is controlled as a function of depth based on the set-point of a single control by mapping the selected set point onto a complete set of gain adjustment data that specifies the gain at each of N depths. The gain at each of those depths is then adjusted based on the selected set of gain adjustment data.

### BRIEF DESCRIPTION OF THE DRAWINGS

FIG. 1 is prior art default TGC curve that plots gain as a function of depth.

FIG. 2A depicts a set of controls for making gain adjustments at different depths of the image in a prior art ultrasound system.

FIG. 2B depicts a gain adjust curve for a prior art ultrasound system.

FIG. 2C depicts an adjusted gain curve for a prior art ultrasound system.

FIG. 3 is a first embodiment of a user interface for controlling the gain of an ultrasound system at different depths.

FIG. 4 is a family of gain adjust curves for the first embodiment.

FIG. 5 is a family of adjusted gain curves for the first embodiment.

FIG. 6 is a flowchart that depicts one approach for building an adjusted gain curve.

FIG. 7 is a block diagram of an ultrasound system that builds and uses an adjusted gain curve.

FIG. 8A is a second embodiment of a user interface for controlling the gain of an ultrasound system at different depths.

FIG. 8B is a third embodiment of a user interface for controlling the gain of an ultrasound system at different depths.

FIG. 9 is a fourth embodiment of a user interface for controlling the gain of an ultrasound system at different depths.

FIG. 10 is an additional family of gain adjust curves for the fourth embodiment.

FIG. 11 is an alternative family of gain adjust curves for the first embodiment.

### DETAILED DESCRIPTION OF THE PREFERRED EMBODIMENTS

The preferred embodiments described herein provide an improved approach for implementing depth-based gain control. They provide a simplified approach for controlling deviations from the default TGC gain curve without requiring individually and independently adjustable gain adjustment controls for each of a plurality of depths.

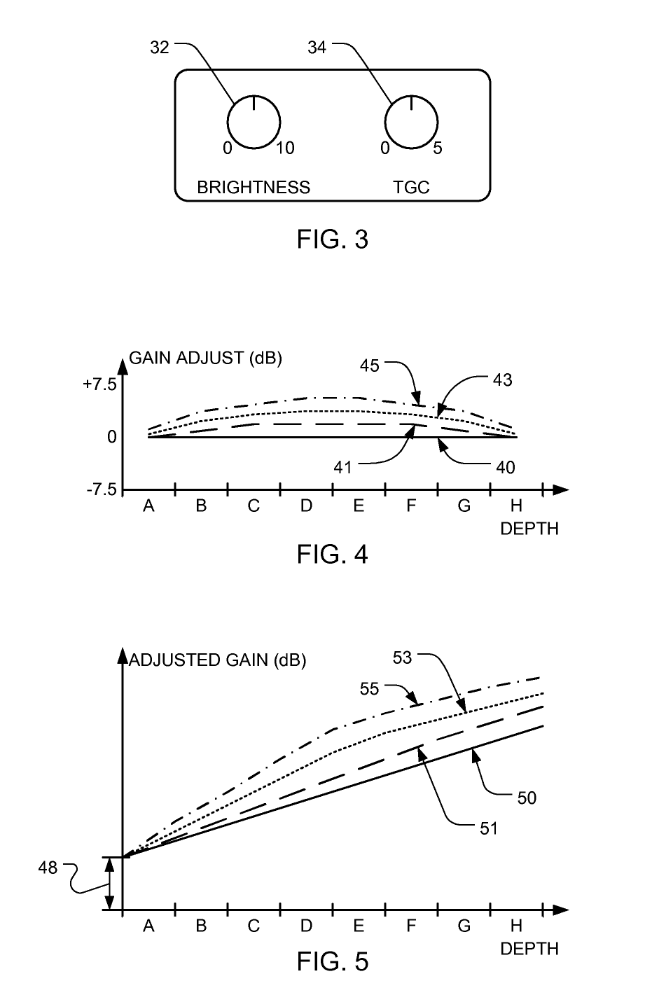

FIG. 3 shows a set of user interface controls for a first embodiment of the invention in which the adjusted TGC curve is controlled using only two controls—a brightness control 32 and a TGC control 34. Note that while these controls 32, 34 are depicted as rotary knobs, persons skilled in the relevant arts will appreciate that a wide variety of alternative user interfaces (including but not limited to sliders, virtual controls on a display screen, etc.) can be substituted therefor.

In this embodiment, different levels of deviations from the default TGC curve for each of many different depths are set simultaneously using a single control 34. This is accomplished by having each position of the TGC control 34 correspond to a complete gain adjustment curve that specifies the deviations from the default gain curve at each depth. FIG. 4A is a schematic representation of four such curves 40-45, and one of those curves is selected based on the position of the TGC control 34. Thus, when the user sets the TGC control 34 to a particular position, it causes the system to select a complete gain adjustment curve from a predetermined family of gain adjustment curves, where each curve in the family specifies what the deviations from the default gain curve is for all depths. For example, setting the TGC control 34 to position 0 would select gain adjustment curve 40, setting the TGC control 34 to position 1 would select gain adjustment curve 41, etc. Note that while FIG. 4 depicts only a small number of curves for clarity, in practice it is preferable to use a larger number of curves (e.g. 8, 16, or 32) to provide a finer degree of control to the end user. Note also that while FIG. 4 depicts that the image is divided into eight depth zones A-H, the depth zones may be divided into smaller increments (e.g., into 16 or 32 depth zones) or larger increments (e.g., into 4-6 depth zones). The particularly gain adjustment curve that is selected by the user based on the position of the TGC control 34 is referred to herein as the “selected gain adjustment curve.”

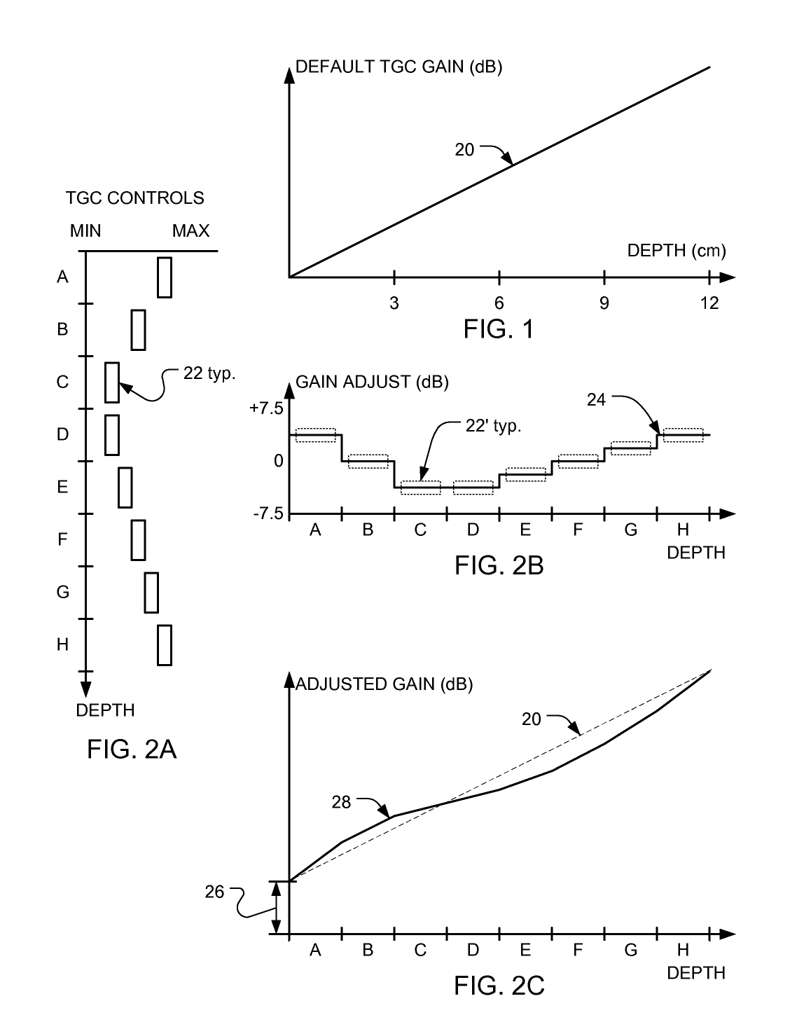

The selected gain adjustment curve is used by the system together with other controls to generate an adjusted TGC gain curve that determines the gain used at each depth. The determination of gain is preferably implemented by adding the gain adjustment from the selected gain adjustment curve to the default TGC gain curve 20 (shown in FIG. 1) for each depth. Preferably, a second control (i.e., the brightness control 32, shown in FIG. 3) is also provided to offset the entire adjusted TGC gain curve up and down by a user-selectable amount.

FIG. 5 shows what four different adjusted TGC gain curves 50, 51, 53, 55 would look like depending on which of the four gain adjustment curves 40, 41, 43, or 45 (shown in FIG. 4) was selected based on the position of the TGC control 34 (shown in FIG. 3). For example, setting the TGC control 34 to position 0 would cause the selection of gain adjustment curve 40 in FIG. 4. Because curve 40 is flat (i.e. 0 dB of gain adjustment at all depths), the resulting adjusted TGC gain curve would be curve 50 in FIG. 5. Similarly, setting the TGC control 34 to position 1 (in FIG. 3) would cause the selection of gain adjustment curve 41 in FIG. 4, which would cause the adjusted TGC gain curve to resemble curve 51 in FIG. 5. Note that for all the curves 50-55 in FIG. 5, the magnitude of the offset 48 for the entire curve is controlled by the position of the brightness control 32 (shown in FIG. 3).

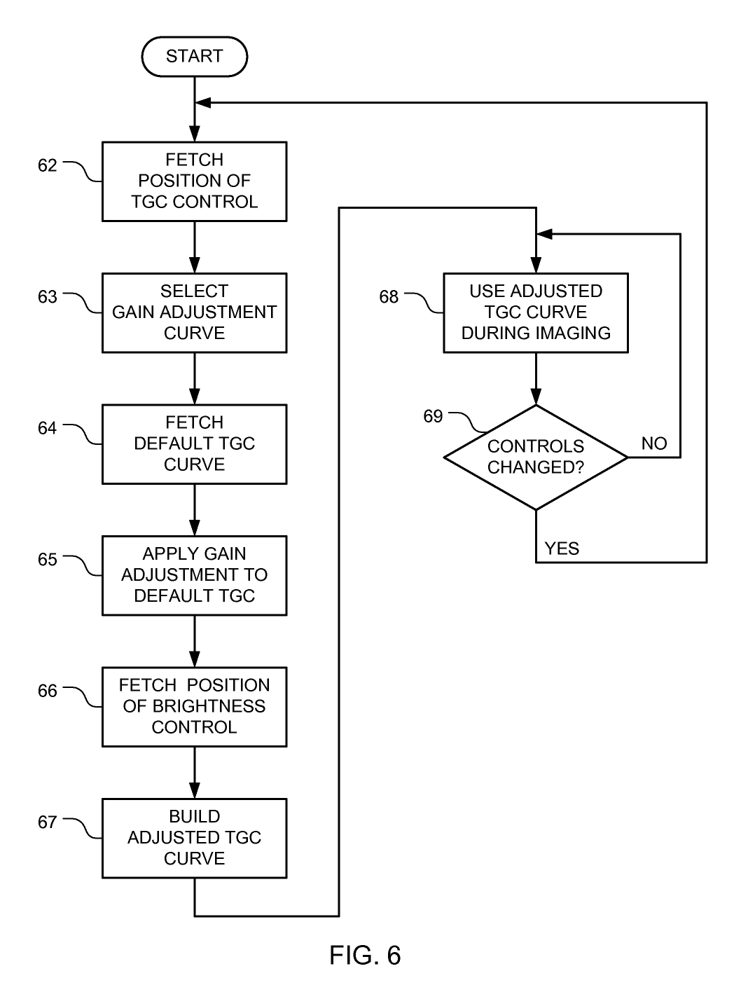

FIG. 6 is a flow chart of one example approach for generating any of the adjusted TGC curves depicted in FIG. 5 based on the position of the brightness control 32 and the TGC control 34 (both shown in FIG. 3). In this example, the image has been divided into eight depth bins (i.e., depths A-H), and data is provided for five different gain adjustment curves (i.e., curves 0 through 5). One way to store the gain adjustment data in the system is by using a table stored in memory that specifies a gain adjustment in dB for each of the five curves at each of the eight depth bins. See, for example, Table 1.

TABLE 1

CURVE

| DEPTH | #0 | #1 | #2 | #3 | #4 | #5 |
|---|---|---|---|---|---|---|
| A | −7.50 | −7.32 | −5.97 | −4.50 | −2.97 | −1.04 |
| B | −7.50 | −5.27 | −3.92 | −2.57 | −1.20 | 0.92 |
| C | −7.50 | −3.74 | −2.39 | −1.20 | 0.03 | 2.27 |
| D | −7.50 | −3.03 | −1.50 | −0.09 | 0.92 | 3.21 |
| E | −7.50 | −3.03 | −0.92 | 0.68 | 1.80 | 3.92 |
| F | −7.50 | −3.03 | −0.74 | 0.62 | 2.27 | 4.38 |
| G | −7.50 | −3.03 | −0.74 | 0.62 | 2.27 | 4.86 |
| H | −7.50 | −4.20 | −1.50 | 0.15 | 2.27 | 4.86 |

Depending on the characteristics of the hardware being used, it may be more convenient to deal with the gain by specifying the value of the control signal needed to achieve the desired gain change, instead of specifying the value of gain change itself in dB. For example, in a system where +0.5 V control signal provides a gain of +7.5 dB and a −0.5 V control signal provides a gain of −7.5 dB, Table 2 would represent the same gain changes shown in Table 1, except that the values in Table 2 are specified in mV instead of dB. The remainder of this specification uses this convention, and deals mainly with the gain control signal (in mV) instead of the gain in dB.

TABLE 2

CURVE

| DEPTH | #0 | #1 | #2 | #3 | #4 | #5 |
|---|---|---|---|---|---|---|
| A | −500 | −488 | −398 | −300 | −198 | −69 |
| B | −500 | −351 | −261 | −171 | −80 | 61 |
| C | −500 | −249 | −159 | −80 | 2 | 151 |
| D | −500 | −202 | −100 | −6 | 61 | 214 |
| E | −500 | −202 | −61 | 45 | 120 | 261 |
| F | −500 | −202 | −49 | 41 | 151 | 292 |
| G | −500 | −202 | −49 | 41 | 151 | 324 |
| H | −500 | −280 | −100 | 10 | 151 | 324 |

Returning now to FIG. 6, in step 62 the position of the TGC control 34 (shown in FIG. 3) is fetched by the system to ascertain the set point of the TGC control that was selected by the user. The fetching step may be implemented using any of a variety of well-known user interface techniques that will depend on the particular type of control (e.g. potentiometers, optical encoders, touch screen, etc.) that is used to implement the TGC control 34, as will be appreciated by persons skilled in the relevant arts.

In step 63, one of the gain adjustment curves is selected based on the position of the control that was fetched in step 62. One simple way to implement this selection is to divide the full range of motion of the TGC control 34 (shown in FIG. 3) into N equal subranges, where N is equal to the number of curves stored in memory, and then select the data for curve #0 when the control is positioned in the first subrange, select the data for curve #1 when the control is positioned in the next subrange, etc.

In step 64, the default TGC curve is fetched. Table 3 depicts an example of a suitable default TGC curve that specifies a gain of about 3 dB per centimeter.

TABLE 3

| DEPTH | cm | dB | mV |
|---|---|---|---|
| A | 0 | 0 | 0 |
| B | 1.57 | 4.8 | 321 |
| C | 3.14 | 9.6 | 643 |
| D | 4.71 | 14.5 | 964 |
| E | 6.29 | 19.3 | 1286 |
| F | 7.86 | 24.1 | 1607 |
| G | 9.43 | 28.9 | 1929 |
| H | 11.0 | 33.8 | 2250 |

Note that the data in Table 3 represents the same 3 dB/cm default TGC function that was depicted by curve 20 in FIG. 1. Of course, persons skilled in relevant art will recognize that since Table 3 represents a linear function, the data contain therein may be computed when needed instead of being stored as data points in a table.

Next, in step 65, the data corresponding to the selected gain adjustment curve (from Table 2) is used to modify the default TGC curve (from Table 3). Table 4 depicts the results of this modification for each of the six curves 0-5. To implement this modification, the gain adjustment at each depth A-H is added to the default TGC curve data at each of those depths to form preliminary adjusted TGC curves, and Table 4 shows what the data for those preliminary adjusted TGC curves would look like when each of the six curves 0-5 is used to modify the default TGC curve. Thus, the data in Table 4 represents the control signals that account for both depth of penetration and the gain adjustment curve that was selected by the user via the TGC control 34 (shown in FIG. 3).

TABLE 4

CURVE

| DEPTH | #0 | #1 | #2 | #3 | #4 | #5 |
|---|---|---|---|---|---|---|
| A | −500 | −488 | −398 | −300 | −198 | −69 |
| B | −179 | −30 | 61 | 151 | 241 | 382 |
| C | 143 | 394 | 484 | 562 | 645 | 794 |
| D | 464 | 762 | 864 | 958 | 1025 | 1178 |
| E | 786 | 1084 | 1225 | 1331 | 1405 | 1546 |
| F | 1107 | 1405 | 1558 | 1648 | 1758 | 1899 |
| G | 1429 | 1727 | 1880 | 1970 | 2080 | 2252 |
| H | 1750 | 1970 | 2150 | 2260 | 2401 | 2574 |

The purpose of the brightness control 32 (shown in FIG. 3) is to increase or decrease the overall brightness of the entire image period by boosting the overall gain for the entire image. In step 66, the position of the brightness control is fetched, and in step 67 the system builds the adjusted TGC curve that will be used for subsequent imaging. This step may be implemented, for example, by the control value that corresponds to the set position of the brightness control to adjust the gain control signal by a constant value at all depths. For example, if the brightness control 32 is set to provide an overall 6 db of gain in a system where 6 db corresponds to a control signal of 400 mV, 400 mV would be added to every data point in Table 4 to yield the data set depicted in table 5. Note that in situations where the control signal would exceed the maximum allowable value for the amplifier, the gain control setting should be set to its maximum, and digital gain can be used to post-process the corresponding pixels in the picture.

TABLE 5

CURVE

| DEPTH | #0 | #1 | #2 | #3 | #4 | #5 |
|---|---|---|---|---|---|---|
| A | −100 | −88 | 2 | 100 | 202 | 331 |
| B | 221 | 370 | 461 | 551 | 641 | 782 |
| C | 543 | 794 | 884 | 962 | 1045 | 1194 |
| D | 864 | 1162 | 1264 | 1358 | 1425 | 1578 |
| E | 1186 | 1484 | 1625 | 1731 | 1805 | 1946 |
| F | 1507 | 1805 | 1958 | 2048 | 2158 | 2299 |
| G | 1829 | 2127 | 2280 | 2370 | 2480 | 2652 |
| H | 2150 | 2370 | 2550 | 2660 | 2801 | 2974 |

In step 68, the adjusted TGC curve is then used for subsequent imaging operations until such time as the controls 32, 34 (shown in FIG. 3) are adjusted, based on the operation of control branch 69. If the controls are adjusted, processing returns to the beginning so that a new adjusted TGC curve can be formed, and then used for imaging.

Due to the interactions between steps 68 and control branch 69, the imaging process is ongoing while the controls are being adjusted, and the operator can see the results of changing the controls 32, 34 (shown in FIG. 3) in real time on the ultrasound machine's display. When the number of stored curves is small (as in the above-described example), adjusting the controls can cause the image to change in large “jumps” when the TGC control 34 (shown in FIG. 3) crosses the boundary between subranges. Optionally, this jumpiness can be eliminated using a variety of approaches that should be apparent to persons skilled in the relevant arts, such as increasing the number of curves so that the size of the subranges will be smaller, or interpolating between the various curves based on the position of the TGC control to generate interpolated gain adjustment curves that correspond to intermediate positions of the TGC control 34. In alternative embodiments, curve fitting may be used instead of interpolation to generate gain adjustment curves that correspond to intermediate positions of the TGC control 34.

Of course, it will be appreciated by persons skilled in the relevant art that a wide variety of alternative approaches for generating the adjusted TGC curve based on the position of those two controls 32, 34 can be readily envisioned, without relying on the Tables 1-5 described in the example above. More specifically, the math is simple enough to generate the adjusted TGC curve by simply fetching the position of the brightness control 32 and the TGC control 34 (both shown in FIG. 3) and computing the values for the adjusted TGC curve at each depth based on those two fetched position.

Note that the above-described example can be modified by dividing the image into a different number of depth bins, either larger or smaller. If desired, a value for the adjusted TGC curve can be computed for each pixel in the image individually based on the depth of the pixel in question (e.g., using interpolation or curve fitting for intermediate points), and the position of the brightness and TGC control. For example, in a system with a 12 centimeter depth of penetration, in which the samples are spaced 0.015 milliliters apart, the 12 centimeter image depth corresponds to 8,000 samples, so an individual gain adjustment may be computed for each of those 8,000 samples.

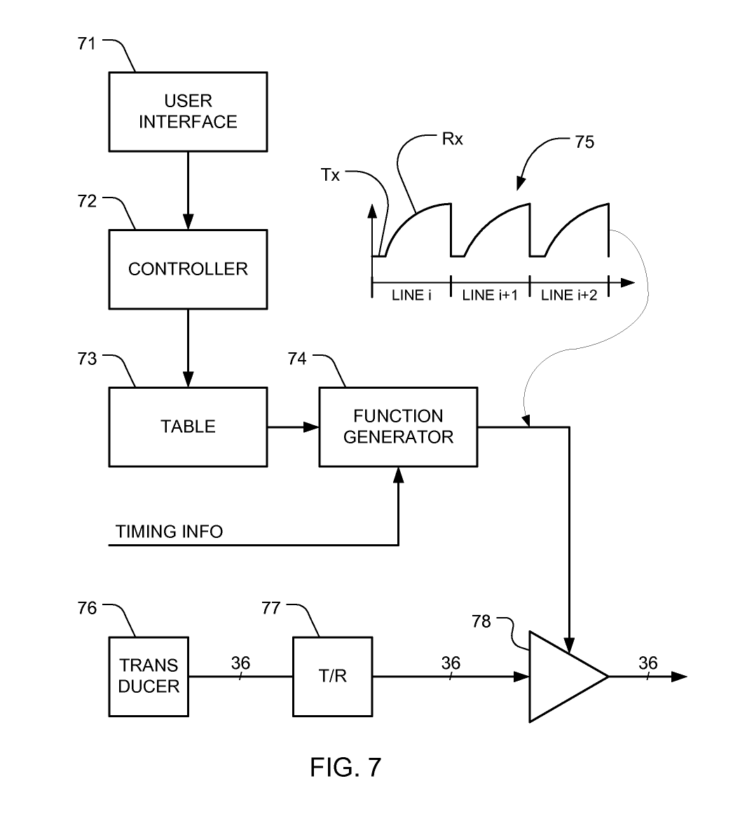

FIG. 7 is a block diagram of a system that computes the adjusted TGC curve and uses that adjusted TGC curve to control gain during ultrasound imaging. In the FIG. 7 embodiment, the ultrasound transducer 76 contains 36 elements, but transducers with any numbers of elements may be used. Although the particular type of ultrasound transducer is not critical, phased array transducers are preferred, especially the type described in U.S. patent application Ser. No. 10/996,816 (filed Nov. 24, 2004) which is incorporated herein by reference. Any conventional ultrasound Transmit/Receive switch 77 may be used to alternately connect the transducer 76 to the transmit pulse generator (not shown) or the receive amplifiers 78. The Texas Instruments VCA2613 is a suitable receive amplifier for this application. Since each VCA2613 contains two amplifiers, 18 VCA2613 devices are needed to amplify the outputs of all 36 transducer elements simultaneously.

The user interface 71 includes the TGC and brightness controls discussed above and may be implemented using any of a variety of conventional approaches. A controller 72 fetches the brightness and the TGC settings from the user interface, computes the shape of the appropriate adjusted TGC curve (e.g., as described above) and stores the resulting data in table 73. One suitable way to implement the table 73 is to load a gain control value for each receive pixel into a table. For example, in a system that uses 8000 pixels per line, a table with 8000 data points may be used to provide an individual gain adjustment for each pixel in a given line.

The function generator 74 generates the adjusted TGC curve repeatedly during the receive cycle and feeds that signal to the gain control input of the amplifier 78 to modify the gain appropriately during different portions of the receive cycle. The function generator 74 is configured to repeatedly output the adjusted TGC curve for each receive interval, for each line of the image in turn, as depicted by waveform 75. For each line, the ultrasound transducer transmits a pulse during periods Tx, after which the system switches to receive mode and receives the return signal corresponding to that pulse using the adjusted TGC curve Rx to modify the gain of the receive amplifier 78. The illustrated waveform 75 depicts this for three consecutive lines of the image i, i+1, i+2, and this continues until a return is received for each line in the image.

One suitable way to implement the function generator 74 is to have the function generator read the gain control value for each receive pixel from the table 73 (e.g., with the read operation controlled by the controller 72 or using DMA), and feed the resulting data stream into a D/A converter. The output of the D/A converter would then be applied to the gain control input of the amplifier 78 in sync with the moment that the corresponding pixel is being received. After all the data points are read, the next pulse is transmitted and the read pointer for the table is reset to start a new receive cycle for the next line in the image. Persons skilled in the relevant arts will recognize that a wide variety of alternative approaches may be used for implementing the repeated generation of the adjusted TGC curve.

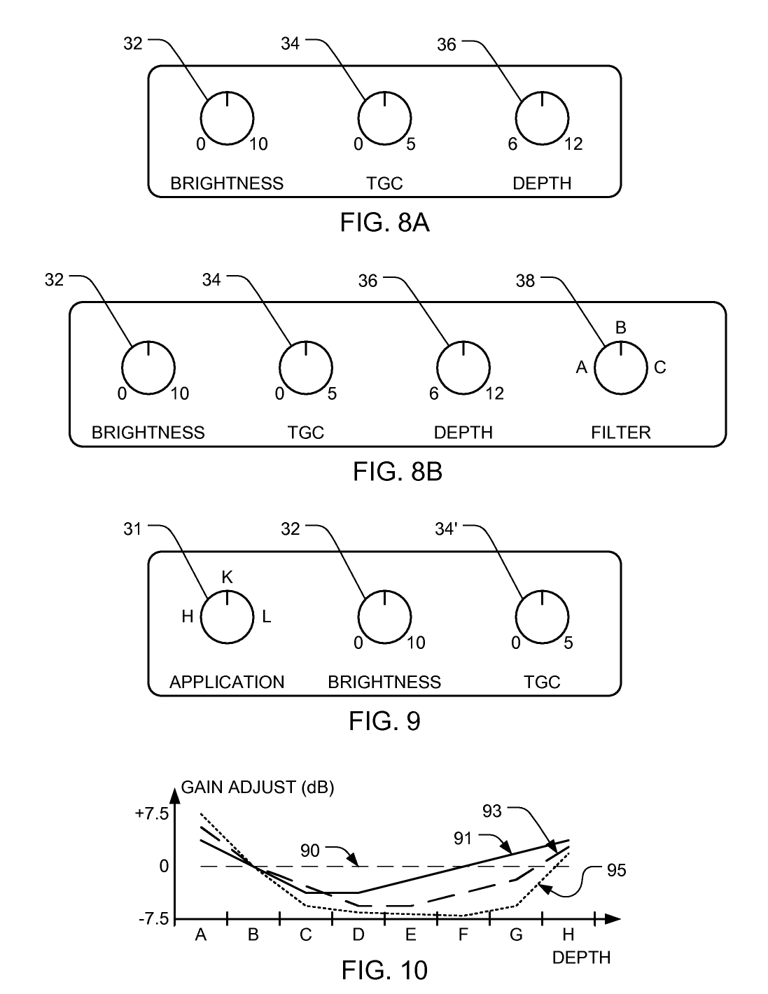

FIG. 8A depicts a second embodiment of a user interface that provides a brightness control 32 and the TGC control 34 that are similar to the first embodiment described above, and adds a depth control 36. In the illustrated embodiment, the depth control can be set between 6 and 12 centimeters. Preferably, the depth control operates by limiting the transmit power when less depth of penetration is needed. This is beneficial for complying with the FDA's ALARA requirements for ultrasound signals.

FIG. 8B depicts a third embodiment of a user interface that provides a brightness control 32, a TGC control 34, and a depth control 36 are similar to the second embodiment described above, and adds a “filter” control 38 that is used to select filter coefficients for the digital post-processing of the raw image data.

FIG. 9 depicts a fourth embodiment of a user interface that provides a brightness control 32 and the TGC control 34′ that are similar to the first embodiment described above, and adds an application control 31. The application control is used to select a family of curves, and the TGC control is used to select one curve from within the selected family. For example, when the application control 31 is set to “H”, the family of curves 50-55 shown in FIG. 5 would be selected, and when the application control 31 is set to “K”, the family of curves 90-95 shown in FIG. 10 would be selected. Other settings of the application control 31 would result in the selection of other families of curves (not shown).

Once the application control 31 has been set to a given position, the TGC control 34′ selects a curve from within the selected family in a manner similar to the way that a single curve was selected from the single family of curves in the first embodiment described above. This application control 31 is useful because one family of curves may not be enough to provide the best images for all possible intended uses due to variations in the anatomy of different target regions or other factors. By providing an application control 31, one family of curves can be optimized for imaging the heart, a second family of curves can be optimized for imaging the kidneys, a third family of curves can be optimized for imaging the lungs, etc. The family of curves that is optimized for the heart, kidneys, or lungs is then selected by switching the application control 31 to H, K, or L, respectively, after which the TGC control 34′ selects a curve from within the selected family. Of course, persons skilled in the relevant arts will recognize that while the above discussion only mentions families of curves that are optimized for heart, kidneys, and lungs, families of curves that are optimized for specific views of those organs or entirely different uses (including both medical and non-medical uses) may also be provided.

The simplified controls described above also make the imaging process more repeatable, since it will be easier to return a given ultrasound machine to a previous state of control settings. For example, a supervisor would be able to instruct the operator to capture an image of a particular subject with the application control, brightness control, and TGC control set to “H”, 5, and 2, respectively. This may be useful, for example, to facilitate the comparison of images obtained from the same patient on different days, or in the context of teaching operators how to use the machine. This repeatability may even be provided across different ultrasound machines that use the above-describe techniques to specify the settings of the machine that was used to capture the image (in a manner analogous to the way that focal length, f-stop, and shutter speed specify a camera's settings in the context of photography).

In an alternative embodiment, instead of selecting the family of curves based on the position of a switch, the family may be selected based on the type of transducer/probe that is hooked up to the system (assuming that an appropriate probe identification approach is implemented).

Optionally, a depth control (similar to one discussed above in connection with the second embodiments) and/or a filter coefficient selector (similar to one discussed above in connection with the second embodiments) may be added to this embodiment.

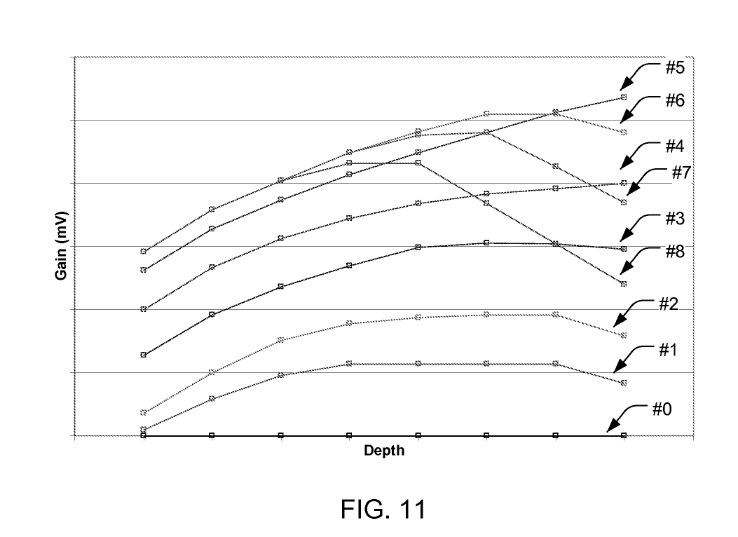

Table 6 is a set of data for an alternative set of gain adjust data which may be substituted for the data set forth above in Table 2, and FIG. 11 is a graph of the set of gain adjust curves that correspond to this data. This particular set of data is specified in mV, for a system where a 300 mV control signal provides a gain of +13.33 dB.

TABLE 6

| | CURVE | | | | | | | | |
|---|---|---|---|---|---|---|---|---|---|
| DEPTH | #0 | #1 | #2 | #3 | #4 | #5 | #6 | #7 | #8 |
| A | 0 | 5 | 18 | 64 | 100 | 131 | 146 | 146 | 146 |
| B | 0 | 29 | 50 | 96 | 133 | 164 | 179 | 179 | 179 |
| C | 0 | 48 | 76 | 118 | 156 | 187 | 202 | 202 | 202 |
| D | 0 | 57 | 89 | 135 | 172 | 207 | 224 | 224 | 216 |
| E | 0 | 57 | 94 | 149 | 184 | 224 | 241 | 238 | 216 |
| F | 0 | 57 | 96 | 153 | 192 | 240 | 255 | 240 | 184 |
| G | 0 | 57 | 96 | 152 | 196 | 256 | 255 | 213 | 152 |
| H | 0 | 42 | 79 | 148 | 200 | 268 | 240 | 185 | 120 |

Note that for this data, the gain for curves 6, 7, and 8 decreases for deeper depths. This is useful for dimming the deepest parts of the image in cases when the relevant portions of the anatomy are all contained in the shallower portions.

1. A method of controlling the gain in an ultrasound system, the method comprising the steps of:
selecting, based on a single set-point of a first control, a set of gain adjustment data from a group of sets of gain adjustment data, wherein all the sets of gain adjustment data in the group are optimized for use during imaging of a first anatomical region wherein each set of gain adjustment data in the group specifies a gain at each of N depths, and wherein the group contains at least 4 sets of gain adjustment data and N is at least 3; and
adjusting the gain at each of the N depths based on the set of gain adjustment data selected in the selecting step.

2. The method of claim 1, wherein the first control is a rotary knob, and the single set-point of the first control is an angular position of the rotary knob.

3. The method of claim 1, wherein the gain adjustment data specifies deviations from a linear TGC curve.

4. The method of claim 1, wherein each of the sets of gain adjustment data contains a data point for each of the N depths.

5. The method of claim 1, wherein each of the sets of gain adjustment data comprises an equation that specifies gain as a function of depth.

6. The method of claim 1, wherein the group contains at least 5 sets of gain adjustment data and N is at least 8.

7. The method of claim 1, further comprising the steps of:
selecting an overall gain based on a single set-point of a second control; and adjusting the gain at each of the N depths based on the overall gain.

8. The method of claim 7, wherein the group contains at least 5 sets of gain adjustment data and N is at least 8.

9. The method of claim 7, further comprising the step of selecting, based on a set-point of a third control, one of a plurality of groups of sets of gain adjustment data, wherein a first group within the plurality of groups contains data optimized for use during imaging of the first anatomical region and a second group within the plurality of groups contains data optimized for use during imaging of a second anatomical region that is different from the first anatomical region.

10. An improved ultrasound system for obtaining an image of a subject by beaming ultrasound energy into a target region, detecting portions of the ultrasound energy reflected from the target region, and processing the detected energy into an image, wherein the improvement comprises a gain control system that has:
a first gain control that selects a set of gain adjustment data from a group of sets of gain adjustment data based on a first single set-point selected by a user, wherein all the sets of gain adjustment data in the group are optimized for use during imaging of a first anatomical region wherein each set of gain adjustment data in the group specifies a gain at each of N depths, and wherein N is at least 3; and
a second gain control that selects an overall gain based on a second single set-point selected by a user,
wherein the ultrasound system adjusts the gain for pixels at each of the N depths based on the set of gain adjustment data selected by the first gain control, and also adjusts the gain for all pixels in the image based on the overall gain selected by the second gain control.

11. The improved ultrasound system of claim 10, wherein the first gain control comprises a rotary knob, and the first single set-point of the first gain control is an angular position of the rotary knob.

12. The improved ultrasound system of claim 10, wherein the group contains at least five sets of gain adjustment data and N is at least 8.

13. The improved ultrasound system of claim 10, wherein the gain control system also has a third control that selects one of a plurality of groups of sets of gain adjustment data based on a third single set-point selected by a user, wherein each group within the plurality of groups includes a first group of data optimized for use during imaging of the first anatomical region and a second group of data optimized for use during imaging of a second anatomical region that is different from the first anatomical region, and wherein the ultrasound system uses the selected group to implement gain adjustments.

14. The improved ultrasound system of claim 13, wherein each group within the plurality of groups contains at least five sets of gain adjustment data, and wherein N is at least 8.

15. A method of controlling the gain in an ultrasound system, the method comprising the steps of:
selecting gain adjustment data for each of N depths based on a single set-point of a first control, where N is at least 3, wherein (a) setting the first control to a first set-point selects a first set of gain adjustment data {A₁, A₂, . . . A_{N}}, for use at each of the N depths, respectively, (b) setting the first control to a second set-point selects a second set of gain adjustment data {B₁, B₂, . . . B_{N}}, for use at each of the N depths, respectively, and (c) setting the first control to a third set-point selects a third set of gain adjustment data {C₁, C₂, . . . C_{N}}, for use at each of the N depths, respectively, and
adjusting the gain at each of the N depths based on the set of gain adjustment data selected in the selecting step,
wherein the first set of gain adjustment data, the second set of gain adjustment data, and the third set of gain adjustment data are all optimized for use during imaging of a first anatomical region.

16. The method of claim 15, where N is at least 8 and wherein (d) setting the first control to a fourth set-point selects a fourth set of gain adjustment data {D₁, D₂, . . . D_{N}}, for use at each of the N depths, respectively, and (e) setting the first control to a fifth set-point selects a fifth set of gain adjustment data {E₁, E₂, . . . E_{N}}, for use at each of the N depths, respectively and
wherein the fourth set of gain adjustment data and the fifth set of gain adjustment data are all optimized for use during imaging of the first anatomical region.

17. The method of claim 15, further comprising the steps of:
selecting an overall gain based on a single set-point of a second control; and
adjusting the gain at each of the N depths based on the overall gain.

18. The method of claim 15, wherein gain transitions between each of the N depths are smooth.

19. An improved ultrasound system for obtaining an image of a subject by beaming ultrasound energy into a target region, detecting portions of the ultrasound energy reflected from the target region, and processing the detected energy into an image, wherein the improvement comprises a gain control system having:
a first gain control that, based on a single set-point selected by a user, simultaneously adjusts the gain at each of N depths, where N is at least 3, wherein (a) setting the control to a first set-point selects a first set of gain adjustments {A₁, A₂, . . . A_{N}}, for use at each of the N depths, respectively, (b) setting the control to a second set-point selects a second set of gain adjustments {B₁, B₂, . . . B_{N}}, for use at each of the N depths, respectively, and (c) setting the control to a third set-point selects a third set of gain adjustments {C₁, C₂, . . . C_{N}}, for use at each of the N depths, respectively, wherein the first set of gain adjustment data, the second set of gain adjustment data, and the third set of gain adjustment data are all optimized for use during imaging of a first anatomical region, and
a second gain control that adjusts the overall gain at all of the N depths by an amount that depends on a set-point of the second control that is selected by a user.

20. The improved ultrasound system of claim 19, where N is at least 8, and wherein (d) setting the control to a fourth set-point selects a fourth set of gain adjustments {D₁, D₂, . . . D_{N}}, for use at each of the N depths, respectively, and (e) setting the control to a fifth set-point selects a fifth set of gain adjustments {E₁, E₂, . . . E_{N}}, for use at each of the N depths, respectively, and wherein the fourth set of gain adjustment data and the fifth set of gain adjustment data are all optimized for use during imaging of the first anatomical region.

21. The improved ultrasound system of claim 19, wherein gain transitions between each of the N depths are smooth.

22. The method of claim 1, wherein the first anatomical region is a heart.

23. The system of claim 10, wherein the first anatomical region is a heart.

24. The method of claim 15, wherein the first anatomical region is a heart.

25. The system of claim 19, wherein the first anatomical region is a heart.
